(19) United States
(12) Patent Application Publication
(10) Pub. No.: US 2008/0105269 A1
(43) Pub. Date: May 8, 2008

(54) SAFETY RESTRAINT AND RELATED METHOD OF USE

(75) Inventor: Michelle L. Rhodes, Grand Rapids, MI (US)

Correspondence Address: WARNER NORCROSS & JUDD LLP, 900 FIFTH THIRD CENTER, 111 LYON STREET, N.W., GRAND RAPIDS, MI, 49503-2487, US

(73) Assignee: SPECTRUM HEALTH HOSPITALS, Grand Rapids, MI (US)

(21) Appl. No.: 11/933,653

(22) Filed: Nov. 1, 2007

Related U.S. Application Data
(60) Provisional application No. 60/864,346, filed on Nov. 3, 2006.

(51) Int. Cl. A61F 5/37

(52) U.S. Cl. 128/870

(57) ABSTRACT

A safety restraint for restraining a patient includes a main body resembling an animal or character. The main body includes a patient facing side defining a pocket, within which a pod having particulate matter may be placed to provide warming or soothing comfort to the patient. The main body can include fasteners adapted to engage corresponding table fasteners on a table to secure the main body over the patient. Optionally, a projection resembling another portion of the animal or character can extend from the main body, with additional straps extending from the projection. The straps can resemble yet another portion of the animal or character, and can restrain an appendage of the patient. A related method of using the safety restraint can be provided, which includes placing the restraint over the patient, warming the soothing pod, placing the pod in the pocket, and securing the restraint over the patient.

This application claims benefit of U.S. Provisional Patent Application 60/864,346 filed on Nov. 3, 2006.

### BACKGROUND OF THE INVENTION

The present invention relates to restraints, and more particularly, to restraints for use with medical treatment and medical imaging equipment.

It is often necessary to immobilize or restrain patients undergoing medical treatment or examination. For example, children undergoing medical imaging, such as magnetic resonance imaging, must usually be sedated and/or physically restrained to ensure proper imaging. If the child is not restrained or sedated, there is a risk that the child will move, and the proper images will not be captured or the imaging process may take a significant amount of time.

Conventional restraint systems often appear and feel intimidating for patients, particularly children and anxiety affected adults. Most restraint systems are designed to work in conjunction with a cradle in which, or a bed on which the patient is positioned. Usually, the cradle or bed includes hook and loop fastener strips along its sides. The restraint includes plain, unattractive grey or black straps that cross the cradle or bed, and corresponding hook and loop fasteners at opposite ends of the straps. These straps are placed over a patient on the cradle and fastened to the bed hook and loop fastener system.

An exemplary construction of such a restraint system is used in a pediatric imaging examination. In this construction, an infant is placed in a cradle board, which defines a recess generally in the shape of the infant's body and head. As shown in FIG. 1, to hold the infant's head in a fixed position, one end of a restraining strap is fastened to hook and loop fasteners on one side of the cradle, crossed over the infant's head, and snugly secured to hook and loop fasteners on the other side of the cradle. Accordingly, the infant's head is strapped in place and immobilized for imaging. A patient's arms can be immobilized in a similar manner with another strap transversely secured to the cradle or bed.

Frequently, the impersonal, unfriendly restraints and the accompanying immobilization of a patient cause that patient and their family to undergo anxiety. This, coupled with the already uncomfortable treatment or imaging procedure, can be traumatic.

### SUMMARY OF THE INVENTION

The aforementioned problems are overcome by the present invention which provides a safety restraint that resembles an animal or character and defines a pocket in which a warming device is placed to provide soothing comfort to a patient as the patient is restrained.

In one embodiment, the pocket can be located adjacent an inner, patient facing surface of the restraint so that it faces a patient's body. Accordingly, when the warming device is placed in the pocket, it can provide soothing warmth and pressure to the patient.

In another embodiment, the restraint can include a main body portion shaped in the form of an animal's body. From the corners of the main body portion, appendages resembling feet or paws, can extend. From a central region of the main body portion, a projection resembling the animal's head can extend.

In yet another embodiment, the projection can include straps extending from it which resemble ears of the animal or character. For example, when the restraint resembles a rabbit or dog, the straps of the restraint can be configured to resemble long, floppy ears of these animals. These straps can include fasteners compatible with those of a cradle or bed to which the restraint is secured. Alternatively, the straps can include fasteners that attach the straps to one another, to each other, and/or to the main body portion. The straps can be of a length sufficient to secure a patient's hands or arms to the sides of their body or above their heads as required for treatment or imaging.

In a further embodiment, the restraint can be constructed from an easily washable and/or sterilizable material, optionally enhanced with an internal batting that makes the restraint warm and soft. Optionally, the material can be waterproof and/or breathable.

In another aspect, a method of using the safety restraint is provided, that method including: providing the safety restraint; warming a soothing pod; placing the soothing pod in a pocket defined by the safety restraint, securing the safety restraint over a patient; optionally restraining appendages of the patient with straps of the restraint; and optionally performing a medical examination or treatment.

The present invention provides a comforting medical restraint that can reduce the anxiety of patients with which it is used. Where the restraint is used with children, it can also provide reassuring comfort by way of the restraint character or animal embracing or hugging the child. Moreover, when outfitted with a pocket that stores a warming device, the restraint can mimic the calming, soothing effect of a human's touch on the patient to provide reassurance and comfort while the patient is immobilized. In turn, this can reduce patient movement, and thereby improving treatment or examination efficiency.

These and other objects, advantages and features of the invention will be more readily understood and appreciated by reference to the detailed description of the invention and the drawings.

### BRIEF DESCRIPTION OF THE DRAWINGS

FIG. 1 is perspective view of a restraint and a table of the prior art;

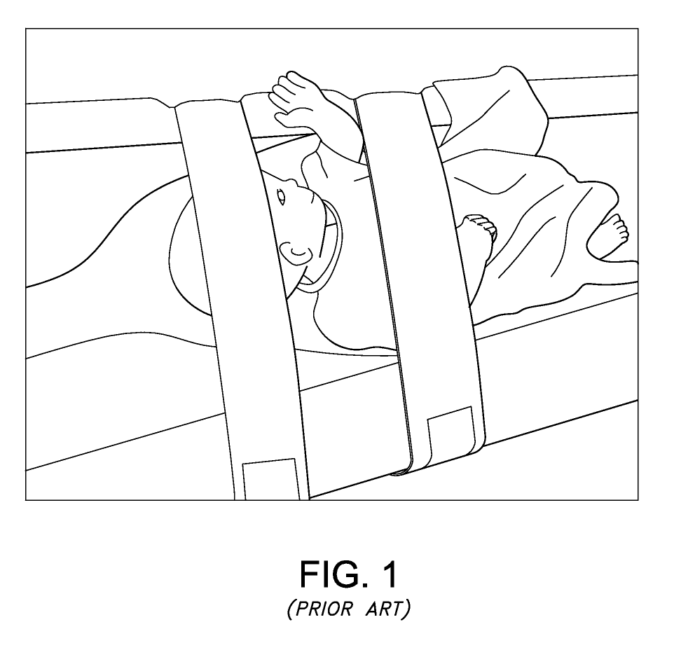

FIG. 2 is a top view of a safety restraint of the present invention;

FIG. 3 is bottom view thereof;

FIG. 4 is a side view thereof showing a through hole defined thereby;

FIG. 5 is a side view thereof showing the through hole having restraining straps placed in the through hole;

FIG. 6 is a side view thereof showing the through hole having a restraining band placed in the through hole;

FIG. 7 is a cross sectional view of material from which the safety restraint can be constructed;

FIG. 8 is a view of a soothing pod that can be stored in the pocket;

FIG. 9 is a view of an alternative, disassembled soothing pod;

FIG. 10 is a view of the safety restraint being partially placed over a patient;

FIG. 11 is a view of the safety restraint being secured over the patient;

FIG. 12 is a view of a strap of the safety restraint being secured to an arm of the patient;

FIG. 13 is a view of another strap of the safety restraint being secured to another arm of the patient, and subsequently to a corresponding fastener strip on a table;

FIG. 14 is a view of another strap of the safety restraint being secured to another arm of the patient, and subsequently to the opposite strap to restrain the arms of a patient in an upward extended position;

FIG. 15 is a view of a projection of the safety restraint in a folded over position, adjacent the main body, away from a face of the patient; and

FIG. 16 is a view of an alternative embodiment of the safety restraint.

### DETAILED DESCRIPTION OF THE INVENTION

### I. Overview

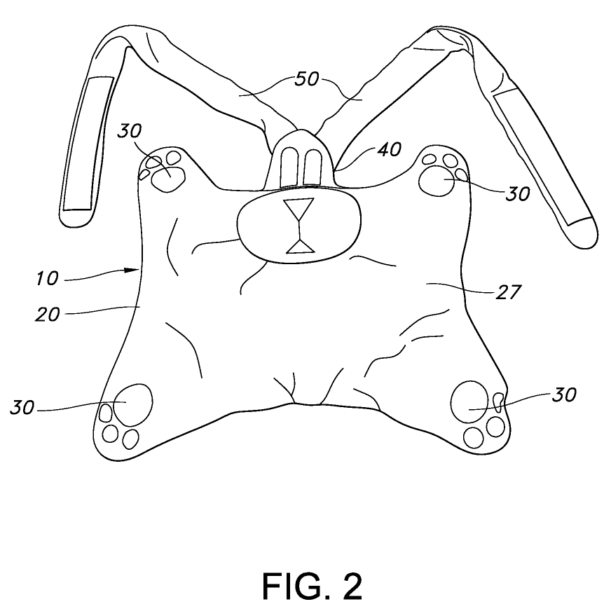

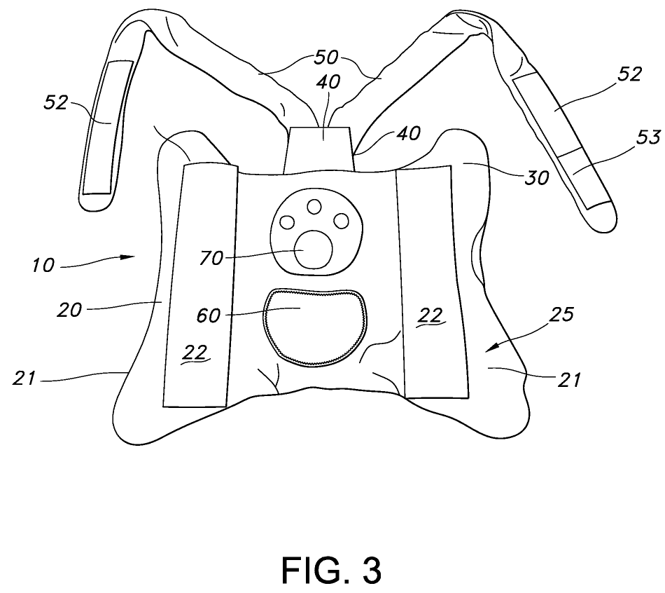

A safety restraint in accordance with an embodiment of the present invention is shown in FIG. 2-3, and generally designated 10. The safety restraint generally includes a main body portion 20 from which appendages 30 and projection 40 extend. To the projection 40 and/or the appendages 30 and/or the main body portion 20, straps 50 can be secured. Further, the safety restraint can define a pocket 60 adapted to store a soothing pod 70.

The safety restraint can include a variety of fasteners, such as hook and loop fasteners (e.g., Velcro®), snaps, clips, conventional hook an loop fasteners, buttons, and other fasteners, compatible with corresponding fasteners of a table with which the restraint is used. As used herein, “table” includes a bed, a Papoose board, a cradle, a support board, and/or any fixture designed to support a patient.

The present embodiment is described in connection with a safety restraint for use with a pediatric cradle to immobilize a child who must undergo an imaging examination, for example, an MRI or an X-Ray examination. The safety restraint, however, can be used with teens, adults and the elderly in a variety of treatments and examination procedures, and with or without a table. For example, the safety restraint is suitable as: (a) a cosmetic improvement, arm restraint and safety device when used with a strap that reaches around an imaging table; (b) an arm restraint when it is wrapped around a patient while a parent holds the patient as an IV is started on the patient; or (c) a restraint when it is placed around the arms of a child while the arms are behind the child's back and the child lays directly on the strap for additional restraint. In addition, the safety restraint can be used in connection with dental examinations or procedures to immobilize children or anxious adults; in connection with medical treatment for patients afflicted with Alzheimer's, paranoia or other mental diseases or disorders; or in connection with pediatric cancer treatment.

### II. Construction

The components of the safety restraint 10 will now be described in more detail with reference to FIGS. 2-9. As shown in FIG. 2 the safety restraint is constructed to resemble an animal, such as a rabbit or a dog. The safety restraint can be modified to resemble any animal or character as desired. As used herein, “resembling and animal” or “portion of an animal” includes resembling an animal, a character, a person, or a well known object or feature, or a portion thereof.

In the construction, the main body portion 20 can include appendages 30 at each of the four corners of the main body portion. These appendages can resemble legs or paws of the simulated animal. In general, the main body portion 20 can be of a width sufficient to span a table or cradle with which the safety restraint is used, while still firmly securing to the fasteners associated with the table. In one embodiment, the main body portion can include a functional width of about 20 inches, with the total width (including appendages) being about 24 inches. The functional length can be about 15 inches, with the total length being about 24 inches, including the projection and appendages. Other dimensions can be used as the application requires.

The main body portion 20 also can include hook and loop fastener strips 22 on its reverse, patient facing side 25, those strips designed to fasten to corresponding fastener strips of a table or cradle board. These strips can be of any dimension, but as shown, are about 2 to 3 inches in width. Further, the fastener strips can be oriented anywhere, but as shown, a first strip is positioned adjacent and parallel to one opposing side 21 and a second strip 21 is positioned adjacent and parallel to the other opposing side of the main body 20. When used in connection with a table, the strips can be matched in size and type to corresponding fasteners on the table.

The main body portion 20, as shown in FIGS. 3-6, can be constructed from one or more sheets 80. In particular, the main body portion can include a first sheet 82 that generally faces a patient, and a second sheet 84, that can form an exterior of the restraint 10. The sheets 80 can be joined together at the opposing ends of the main body 20, for example with stitching or fasteners or other suitable connection devices. Each sheet 82 and 84 may individually be constructed from component upper and lower sheets, 81, 83 and 85, 86. These component sheets can be constructed from, for example, woven fabrics, laminates and/or synthetic or natural materials. One suitable material is a polyurethane laminate. Any thickness of the laminate can be used, for example 1 mil to 5 mil, and optionally 2 mil. The material can be colorfast, waterproof, water resistant and/or breathable. In one embodiment, the material is machine washable and dryable, and can withstand repeated treatment with infection control compositions. This can add to the longevity of the material, and make the restraint easy to sterilize, clean and maintain.

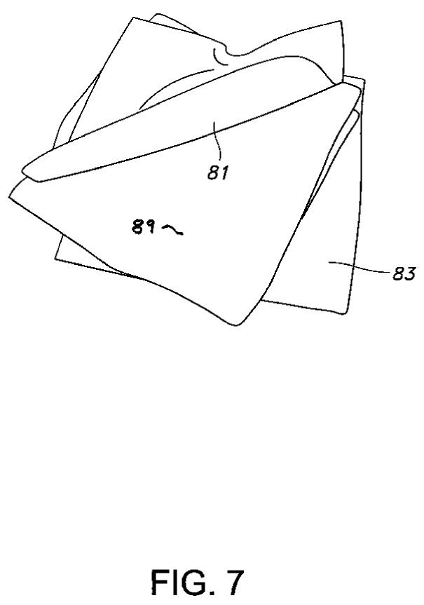

An example of one possible construction of the sheets 80 (as well as other components of the restraint) is shown in FIG. 7. There, the portion of the restraint includes an upper sheet 81, a lower sheet 83 and a batting 89 sandwiched therebetween. The batting can provide added cushioning to make the restraint more soothing and comfortable to a restrained patient, or to add loft to the sheets 80. The sheets 80 and their component sheets, as well as various other parts of the restraint, can be secured together with thread using conventional sewing techniques or other processes such as gluing as desired. The resultant component of the safety restraint can be soft and flexible, and optionally able to conform to a patient's body or appendages as desired.

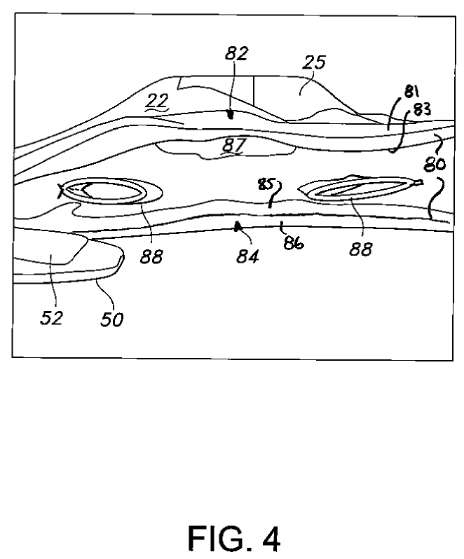

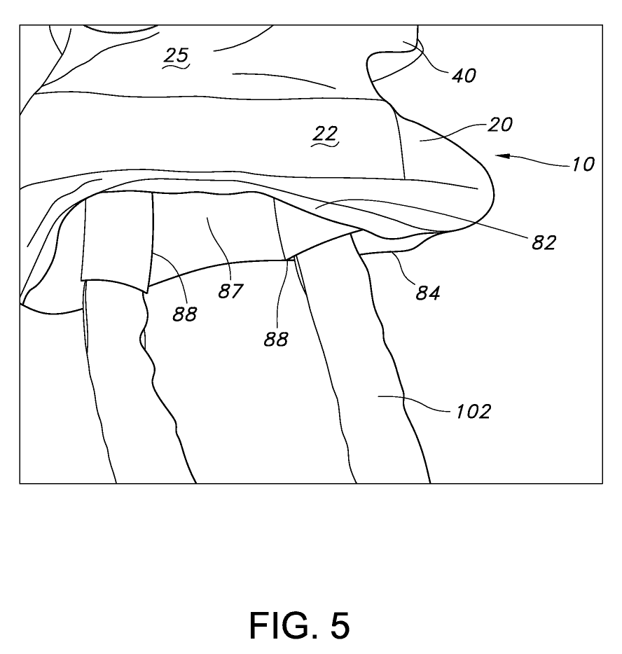

Returning to FIGS. 4-6, between the opposing ends, the sheets 82 and 84 can be unjoined so as to form an open through hole 87. This through hole 87 can be of any size or shape, and can be designed to accommodate a variety of additional straps or bands that assist in restraining the patient. For example, with reference to FIG. 5, the through hole 87 can include additional strap retainer loops 88, which are joined with one or more of the sheets 80 by seams or fasteners. These strap retainer loops 88 can support and/or guide restrain straps 102 within the opening 87. The restraint straps 102 can be placed around a patient in a desired configuration, while holding the safety restraint adjacent the patient.

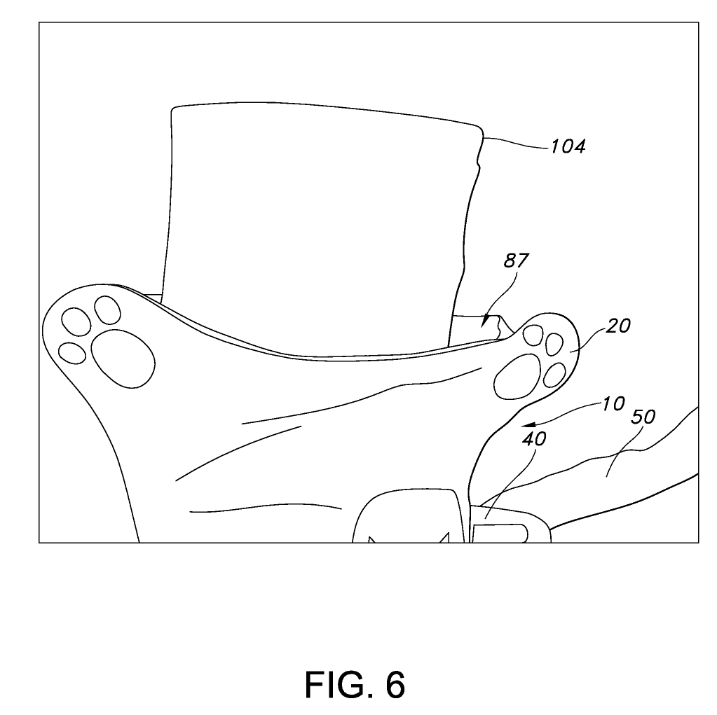

With reference to FIG. 6, a larger restraining band 104 can also be placed through the through hole 87. This band 104 can be placed around a patient in a desired configuration as well. In cases where the safety restraint is used to secure the patient to a table, the band can circumferentiate the table as desired.

To the central region of the main body portion 20, another projection 40 can be secured or integrated as shown in FIGS. 2-3. This projection can be configured to resemble the head of the simulated animal. The projection 40 can include animal features, such as eyes and a nose, which optionally are appliquéd to the projection to minimize parts that can be removed from the safety restraint and possibly cause harm to the child or patient.

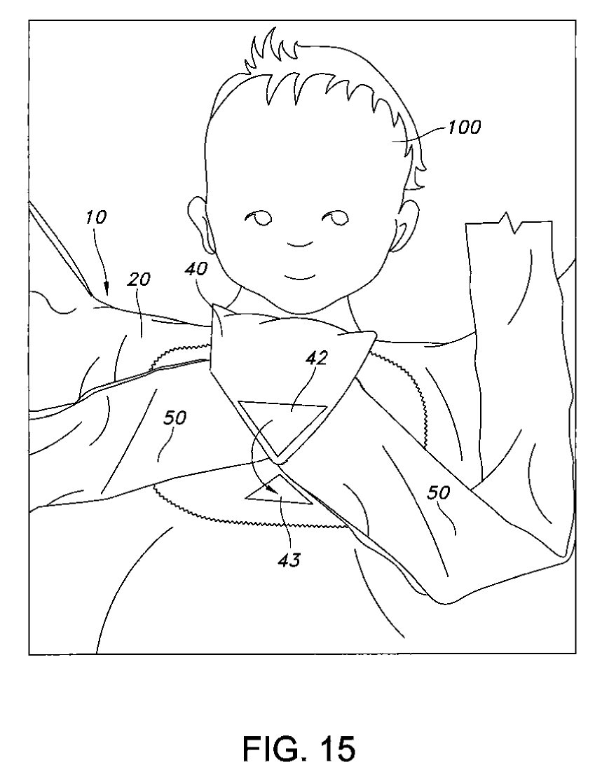

The projection 40 optionally can include features which enable it to be moved from an extended mode, as shown in FIG. 3, to a retracted mode, as shown in FIG. 15. For example, as shown in FIG. 15, the projection can include a projection fastener 42 on its underside, which generally faces the patient. The main body portion 20 can include a complimentary main body portion fastener 43. The projection 40 can be folded over toward the outward side of the main body portion 20 to distance the projection 40 from the patient's face, and minimize inhibition of the patient's breathing. The fastener 42 can engage the complimentary main body portion fastener 43 to secure the projection 40 in the retracted mode so that the projection is held away from a patient's face for whatever period is desired. As desired, the fasteners 42 and 43 can take any shape or form.

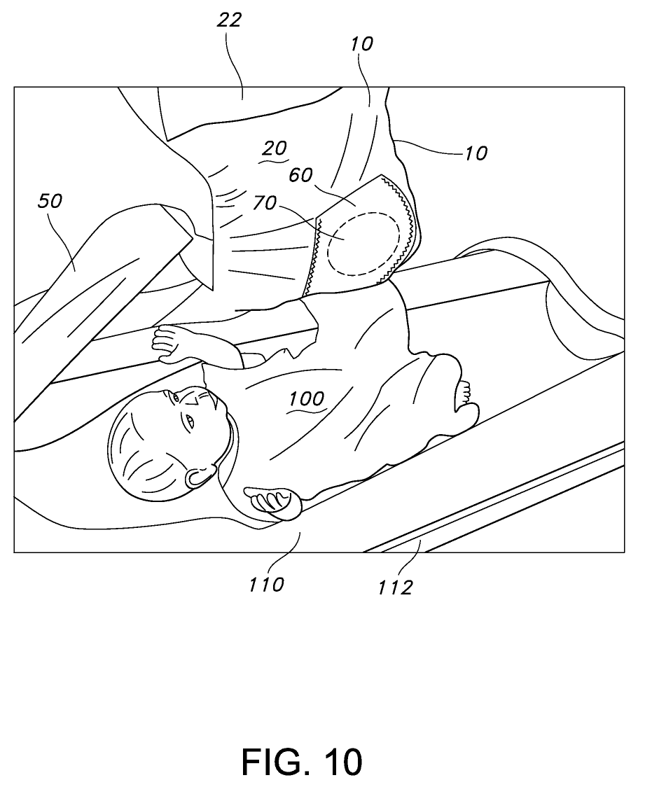

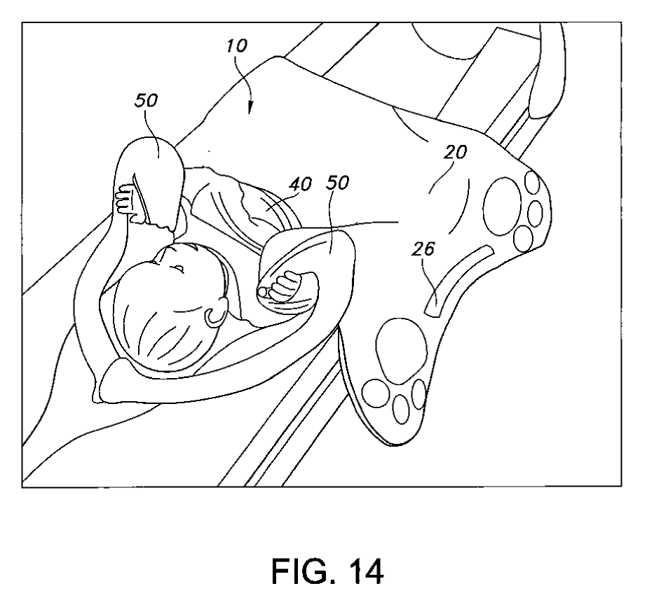

To the projection 40, or optionally the main body portion 20, restraint straps 50 can be secured. With reference to FIGS. 2-3, the restraint straps 50 can be shaped to resemble ears of the animal, or other desired appendages. As shown, the straps 50 can have a length of 10 inches to 30 inches, and optionally can be 27 inches. The width of the straps can range from 1 inch to 5 inches, and optionally can be about 2.5 inches. Secured to the underside of the restraint straps can be a fastener strip, for example a hook and loop fastener strip 52 being about 10 inches to 20 inches, and optionally 15 inches in length, and about 0.5 inches to about 2 inches in width. These hook and loop fastener strips 52 can mate with corresponding fastener strips 112 positioned on the table 110 (FIG. 10), or optionally on corresponding fastener strips 26 (FIG. 14) located on the outward facing surface 27 of the main body portion 20. By securing the fastener strips 52 of the straps 50 to the table 110 or to the main body portion, it is possible to secure a patient's arms to their sides or above their heads as required for treatment or examination.

As shown in FIG. 3, at least one of the fastener strips can include two different types of fasteners. For example, the one strap can include a first hook fastener region 52, and a second, different loop fastener region 53. The second, different loop fastener region 53 can be securable to the hook fastener on the other strap, enabling the straps 50 to be joined with one another, for example, not to a table, to provide yet another restraining configuration for these components.

As shown in FIGS. 3 and 8-10, the safety restraint 10 can also include or define a pocket 60 adapted to store a pod 70. The pocket 60 can be positioned on the reverse or patient facing side 25 of the main body portion 20, and can be of the same or similar shape as the soothing pod as desired. Optionally, the pocket can be constructed from an elastic material to better secure the pod therein.

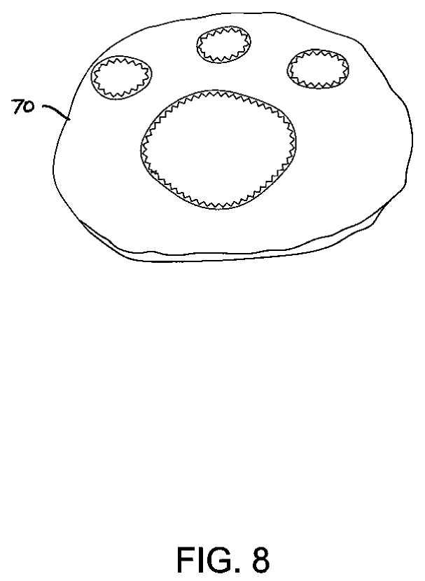

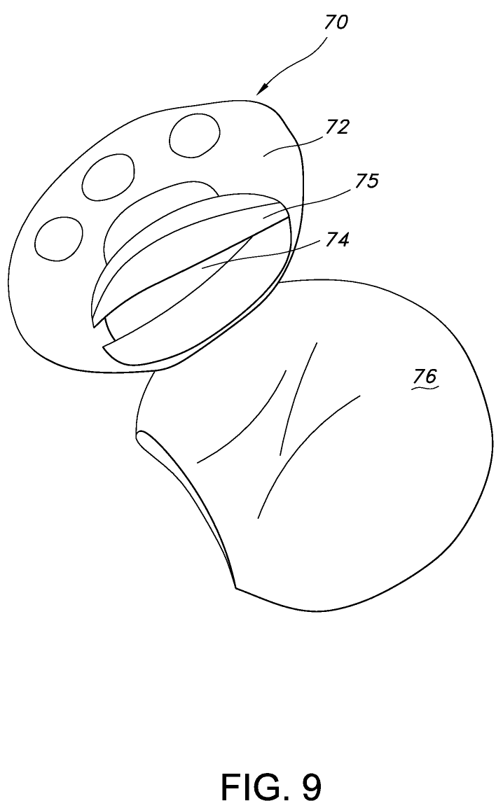

The pod 70 itself, as shown in FIG. 8, can be constructed to form a closed container from the material shown in FIG. 7, or any other acceptable material. Alternatively, the pod can form an open-able container as shown in FIG. 9, which includes a shell 72 defining an opening 74 bounded by a closure 75, such as a hook and loop fastener or other suitable fastener. Within the shell, a pouch 76 can be stored. Optionally, the container can be self-contained and the shell can be absent.

Within the pod 70, for example the pouch 76, a substance can be stored. Optionally, that substance can be of a weight and thermodynamic capacity capable of simulating a human hand when placed in the pocket and over a patient. It has been discovered that this simulation can sooth the patient, and optionally provide a calming effect. In one embodiment, the substance can be flax seed, which is non-hypoallergenic, and is almost odorless upon heating. Other substances such as gels, synthetic fluids, natural substances, such as grains or corns, organic or inorganic pellets, powders, balls and fillers can be substituted as desired. Optionally, the substance can be able to undergo multiple heating events without degrading, can warm to about 60 degrees to about 90 degrees in about one to two minutes, and can remain notably above room temperature without further heating for about 30 minutes to 60 minutes, and optionally about 45 minutes. The amount of the substance can vary so that the weight of the pod 70 weighs from about 0.5 pounds to about 5 pounds, and optionally about 1 pound. Other weights can be used as desired.

### III. Method of Use

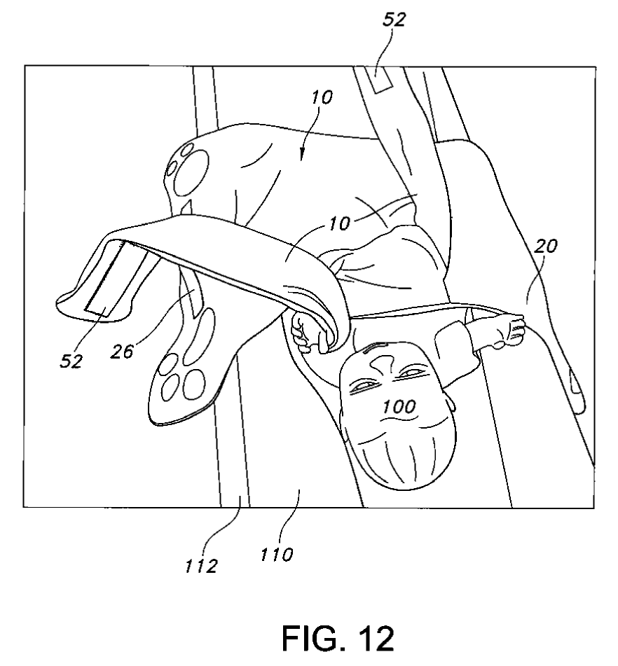

With reference to FIGS. 10-13, a method of using the safety restraint will now be described. As shown in FIG. 10, a patient 100 is placed in a table/cradle board 110, which includes Velcro® fasteners 112 along its sides. The safety restraint 10 is placed over a portion of the patient 100, with the inward facing fastener strips 22 on the patient facing side 25 of the restraint being secured to fasteners 112 on one side of the table 110. The restraint is then placed over the patient, so that the pocket 60 including a pre-warmed pod 70 engages the chest and/or abdomen of the patient. Optionally, the projection 40 can be folded over and secured in a retracted mode as shown in FIGS. 12 and 15 so as not to inhibit the patient's respiration.

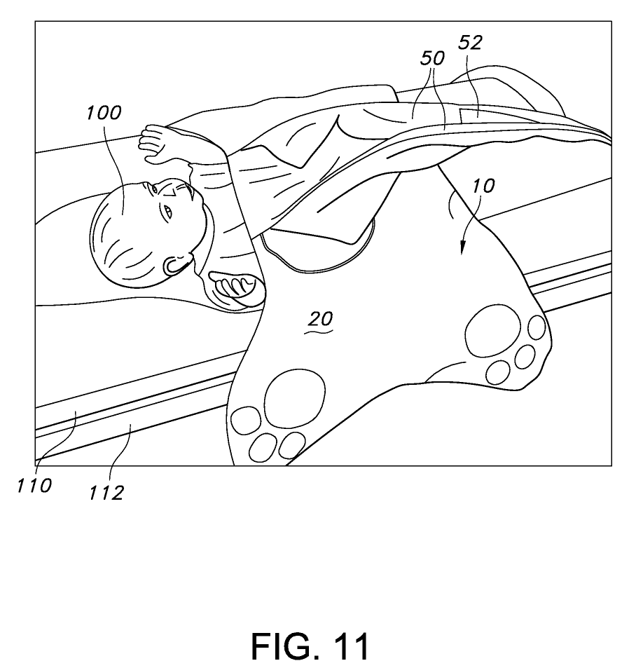

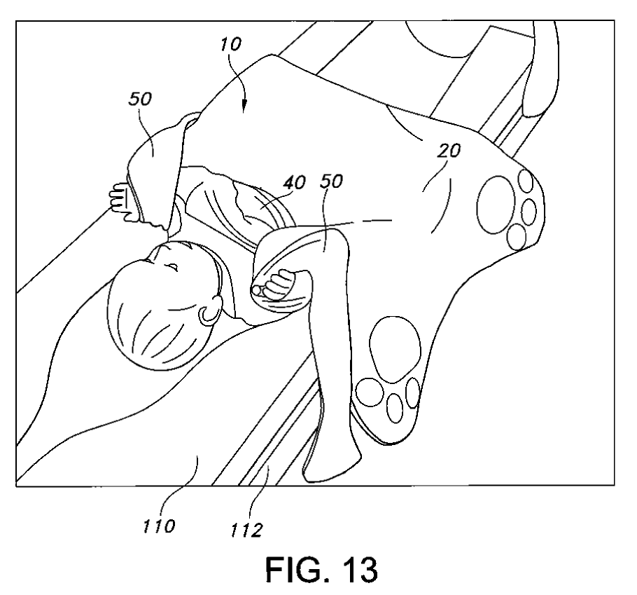

As shown in FIG. 11, the inward facing fastener strips 22 on the opposite side of the restraint are secured to corresponding fastener strips 112 on the cradle board 110. Next, as shown in FIGS. 12 and 13, the straps 50 are secured around the patients arms and positioned so that the fastener strips 52 engage and affix to corresponding fastener strips 112 on the side of the table 110 to secure the patient's arms in place. Although not shown, the straps 50 can be secured to the table fastener strips 112 under the main body portion 20, or alternatively, to optional secondary fastener strips 26 (FIG. 14) joined with the exterior of the main body portion 20. Further alternatively, the straps 50, as shown in FIG. 14 can be secured to one another via connection of the respective fastener strips 52 and 53. Thereafter, the patient can undergo the necessary treatment or examination.

As shown in FIGS. 5 and 6, the safety restraint optionally can be used in the absence of a table. For example, straps 102 or a band 104 can be placed through the through hole 87 (and optionally loops 88), and further around a patient. The straps 102 or band 104 can themselves include corresponding hook and loop fasteners on opposite ends, so that when joined, the opposite ends connect. Optionally, after the straps 102 or band 104 are placed around a portion of a patient, with their ends connected, the straps 50 can be joined with one another, or the straps or band, to restrain the patient. Such a restraining configuration can be useful in the application of an IV to calm and sooth a patient undergoing this sometimes stressful procedure.

The safety restraint 10 can be used in a variety of different environments and in conjunction with a variety of different tables or restraint straps as desired to provide the desired amount of immobilization of the patient, while also providing a soothing, calming effect.

### IV. Alternative Embodiment

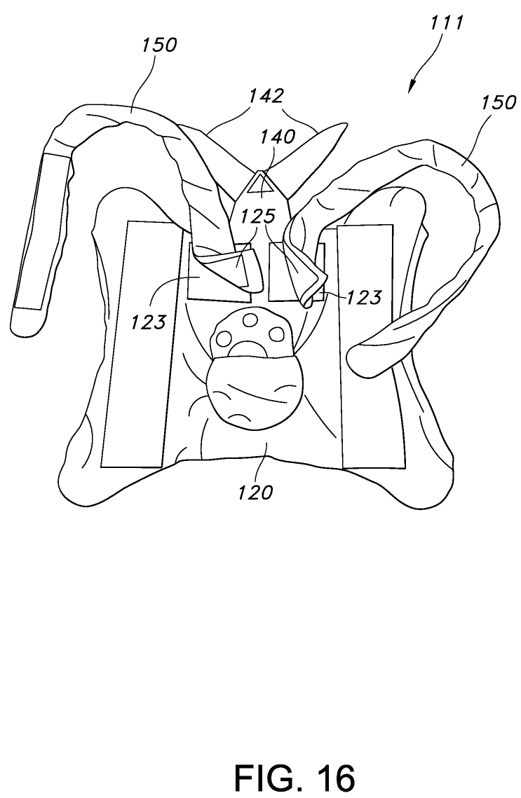

An alternative embodiment of the safety restraint is shown in FIG. 16, and generally designated 111. This alternative restraint is generally the same as the restraint 10 described above, with a few exceptions. First, the main body 120 includes secondary strap fasteners 123, in addition to the primary fasteners 124. To these secondary strap fasteners, the restraining straps 150 are secured via corresponding fasteners 125. A variety of other constructions could be used to secure the straps 150 to the main body 120.

Second, because the projection 140 is not joined with the restraining straps 150, the projection can include additional features 142 joined with it. As shown in FIG. 16, those features resemble ears of the animal; however, those features 142 can resemble other portions of an animal as desired.

The above descriptions are those of the preferred embodiments of the invention. Various alterations and changes can be made without departing from the spirit and broader aspects of the invention as defined in the appended claims, which are to be interpreted in accordance with the principles of patent law including the doctrine of equivalents. Any references to claim elements in the singular, for example, using the articles “a,” “an,” “the,” or “said,” is not to be construed as limiting the element to the singular.

The embodiments of the invention in which an exclusive property or privilege is claimed are defined as follows:

1. A safety restraint for restraining a patient comprising:
a main body portion configured to resemble a portion of an animal, the main body portion including a patient facing side and an outward side, the patient facing side defining a pocket adapted to be positioned immediately adjacent a patient to whom the main body portion is secured;
a projection resembling another portion of the animal extending from the main body portion;
at least two straps extending from at least one of the projection and the main body portion, each strap resembling yet another portion of the animal, the straps adapted to restrain at least one appendage of a patient, each strap including strap fasteners; and
a pod positioned in the pocket, the pod including a particulate material adapted to be heated and to transfer heat and weight to the patient when the main body portion is placed over the patient, whereby the patient is soothed and calmed while being restrained on the table.

2. The safety restraint of claim 1 wherein the projection is in the form of a head of the animal, and wherein each strap resembles an ear of the animal, wherein one strap includes a first hook and loop fastener and a second, different hook and loop fastener, the second, different hook and loop fastener securable to the hook and loop fastener on another strap, whereby the one strap can be secured to the other strap via a hook and loop fastener connection.

3. The safety restraint of claim 1 wherein the projection is movable from an extended mode and a retracted mode, wherein the projection includes a projection fastener, wherein the main body portion includes a complimentary main body portion fastener, wherein the projection fastener engages the complimentary main body portion fastener to secure the projection in the retracted mode so that the projection is held away from a patient's face.

4. The safety restraint of claim 1 wherein the pod includes a resealable opening, an internal cavity and a container, the container containing the organic, particulate matter, the container positioned within the internal cavity, the resealable opening closed to restrain the container with in the internal cavity.

5. The safety restraint of claim 1 wherein the main body includes an upper portion, a lower portion, and opposing sides, wherein the main body includes main body fasteners having a pair of opposing fastener strips, the fastener strips oriented in parallel, one fastener strip being positioned on one opposing side, the other fastener strip being disposed on the other opposing side, the main body fasteners adapted to engage corresponding table fastener strips located on a table, wherein joining the main body fasteners to the corresponding table fastener strips secures the main body portion over the patient to restrain the patient on the table.

6. The safety restraint of claim 1 wherein the particulate matter is flaxseed.

7. The safety restraint of claim 1 wherein the main body portion includes an upper portion, a lower portion, and opposing sides, the main body portion defining a cavity extending transversely from one opposing side to the other to form a continuous through hole, whereby a user places at least one of a secondary strap and a band through the through hole, and around a portion of the patient to increase the restraining effectiveness of the restraint.

8. A safety restraint for restraining a patient on a table comprising:
a main body portion that resembles a portion of an animal, the main body portion including opposing ends and opposing sides, a first sheet and a second sheet joined together at the opposing ends, but unjoined together between the opposing ends so as to form an open through hole, the first sheet defining a pocket, the first sheet including at least two parallel strips of sheet hook and loop fasteners, a first strip positioned adjacent and parallel to one opposing side, a second strip positioned adjacent and parallel to the other opposing side, the strips of sheet hook and loop fasteners adapted to engage corresponding table hook and loop fasteners on the table to secure the main body portion over the patient, each of the first and second sheets being constructed from a water resistant fabric material;
a projection resembling a head of the animal extending from one of the opposing ends;
at least two elongate straps extending from the projection, each strap resembling at least one of an ear and an appendage of the animal, the straps adapted to restrain at least one appendage of a patient, each strap including strap hook and loop fasteners; and
a pod positioned in the pocket, the pod including an organic, particulate material adapted to be heated and to transfer heat and weight to the patient whereby the patient is soothed and calmed while being restrained on the table.

9. The safety restraint of claim 8 comprising corresponding retracting hook and loop fasteners on the projection and the main body portion, the corresponding hook and loop fasteners adapted to fasten to one another and secure the projection in a folded over position, immediately adjacent the second sheet.

10. The safety restraint of claim 9 wherein at least one of the straps resembling at least one of an ear and an appendage of the animal includes a first hook and loop fastener and a second, different hook and loop fastener, the second, different hook and loop fastener securable to the hook and loop fastener on the other strap, whereby the at least one strap can be secured to the other strap via a hook and loop fastener connection.

11. The safety restraint of claim 10 wherein the first sheet and the second sheet are constructed from a water resistant laminate material.

12. The safety restraint of claim 11 wherein the first sheet and second sheet include an internal batting.

13. The safety restraint of claim 12 wherein the pod includes a resealable opening, an internal cavity and a container, the container containing the organic, particulate matter, the container positioned within the internal cavity, the resealable opening closed to restrain the container with in the internal cavity.

14. A method for restraining a patient on a table comprising:
providing a safety restraint including a main body portion that resembles an animal, the restraint defining a pocket on a patient facing surface of the main body portion, the restraint including opposing sides with opposing fastener strips joined with the patient facing surface adjacent the opposing sides, the fastener strips adapted to engage corresponding fasteners on the table, the main body portion including at least two straps adapted to encircle and restrain at least one appendage of a patient, each of the at least two straps including strap fastener strips;
warming a soothing pod;
placing the soothing pod in the pocket;
aligning the soothing pod with at least one of a chest and an abdomen of the patient; and
securing the safety restraint to the table by joining the opposing fastener strips with corresponding fasteners on the table, the restraint being positioned over the patient to embracingly restrain the patient on the table.

15. The method of claim 14 comprising encircling at least one arm of a patient with a strap, and further securing the strap fastener strips of each strap to the corresponding fasteners on the table.

16. The method of claim 15 wherein in the restraint includes a projection extending from the main body portion, wherein the at least two straps extend from the projection, the projection and restraint including corresponding hook and loop fasteners.

17. The method of claim 16 comprising fastening the corresponding hook and loop fasteners to one another to secure the projection in a folded over position, adjacent the second sheet and away from a face of the patient.

18. The method of claim 14 wherein the main body portion includes an upper portion, a lower portion, and opposing sides, the main body portion defining a cavity extending transversely from one opposing side to the other to form a continuous through hole, comprising placing a secondary strap through the through hole and around a portion of the table.

19. The method of claim 14 comprising performing at least one of a medical examination and a medical treatment on the patient as the patient is restrained by the safety restraint.

20. The method of claim 14 comprising encircling each arm of a patient with a strap, and further securing the strap fastener strips of one strap to the strap fastener strip of the other strap.
